(19) United States
(12) Patent Application Publication
(10) Pub. No.: US 2016/0174934 A1
(43) Pub. Date: Jun. 23, 2016

(54) METHOD AND SYSTEM FOR GUIDED ULTRASOUND IMAGE ACQUISITION

(71) Applicant: SHENZHEN MINDRAY BIO-MEDICAL ELECTRONICS CO., LTD., Shenzhen (CN)

(72) Inventors: Longfei CONG, Shenzhen (CN); Jingang KANG, Shenzhen (CN)

(73) Assignee: SHENZHEN MINDRAY BIO-MEDICAL ELECTRONICS CO., LTD.

(21) Appl. No.: 15/056,895

(22) Filed: Feb. 29, 2016

Related U.S. Application Data
(63) Continuation of application No. PCT/CN2013/083768, filed on Sep. 18, 2013.

(51) Int. Cl. A61B 8/00

(52) U.S. Cl. CPC A61B 8/4254; A61B 8/4444; A61B 8/4263

(57) ABSTRACT

An exemplary system includes a navigation system, an imaging system, and a data acquisition and analysis system. The exemplary system provides actively guidance for ultrasound image acquisition based on position information provided by the navigation system at different times (e.g., before and after an interventional procedure), to ensure that image data is collected at the same location within the object of interest (e.g., a target region of the interventional procedure) using the same probe posture (e.g., location and/or orientation).

### TECHNICAL FIELD

The present invention relates to imaging technology, and in particular, to a method and system for providing guided ultrasound image acquisition.

### BACKGROUND

Today, cancer remains one of the most threatening diseases to people in the world. Among the many available treatment alternatives, surgical removal of tumors is still the most important treatment option for cancer patients. However, some patients are not suitable candidates for surgery due to various health-related complications. Therefore, non-surgical treatment options are important for the clinical treatment of these patients. In recent years, interventional therapy has become an important means of treatment for cancer patients. Among the different interventional therapeutic techniques, non-surgical ultrasound interventional therapy has proven to be an effective procedure for treating many cancers, such as liver cancer, lung cancer, thyroid cancer, etc.

Ultrasound imaging is one of the primary image guidance methods for many minimally invasive and interventional procedures. In particular, most needle biopsies and needle-based ablation procedures are guided by ultrasound. The advantages of ultrasound imaging include the real-time imaging capability, low cost, flexibility in its application, and the fact that no ionizing radiation is used. Sometimes, in addition to gray-scale tissue images, a contrast enhanced ultrasound (CEUS) imaging technique is used to obtain contrast images of particular tissue areas that have been injected with a contrast agent.

At the present, when evaluating an interventional procedure performed on a patient, ultrasound images of the affected area of the anatomy are taken before and after the procedure. Medical personnel compare the post-procedure ultrasound images with the pre-procedure ultrasound images to determine whether all tissues in the target area have been removed, and whether the desired safety margin has been achieved. However, due to the lack of suitable location markers in the anatomy and the change in physical appearance of the target area before and after the operation, it is challenging for the medical personnel to accurately assess the conditions of the target area by comparing the ultrasound images that may or may not correspond to the same imaging conditions and/or tissue locations.

### SUMMARY

The embodiments disclosed herein provides methods, systems, computer-readable storage medium, and user interfaces for an ultrasound imaging system that provides real-time guidance for ultrasound image acquisition, in particular, ultrasound image acquisition for the purposes of post-procedure evaluation of a patient's anatomy that has undergone an interventional procedure. In some embodiments, the guided ultrasound image acquisition can also be used in other situations where acquisition and comparison of ultrasound images of the same object of interest (e.g., any animated or inanimate object or portions thereof) at different times (e.g., before and after a physical change has occurred to the object of interest) are desired.

In particular, a guided ultrasound imaging system is used to acquire ultrasound images of a target area in a patient's anatomy both before and after an interventional procedure (e.g., a tumor ablation procedure) is performed on the target area. During the pre-procedure ultrasound image acquisition, the location and posture of the ultrasound probe are tracked via a navigation system (e.g., a magnetic navigation system). The navigation system has a view field (e.g., a magnetic field produced by a magnetic field generator) in which a navigation probe, and optionally, a reference probe, can be detected. In some embodiments, the reference probe is attached to a part (e.g., skin) of the patient near the target area, and the navigation probe is rigidly attached to the ultrasound probe. Thus, the location and posture of the navigation probe relative to the reference probe can be tracked at all times when the ultrasound probe is maneuvered around the patient's body near the target area during image acquisition. After the interventional procedure is performed on the patient, the guided ultrasound imaging system determines the current location of the navigation probe (e.g., the current location relative to the reference probe) and generates a real-time guidance output to assist the operator to reposition the ultrasound probe to a previous location and posture used to obtain a pre-procedure ultrasound image. In some embodiments, once the guided ultrasound imaging system detects that the current position of the ultrasound probe has been realigned (e.g., meeting predefined alignment criteria) with the previous position used to obtain the pre-procedure ultrasound image, a corresponding post-procedure ultrasound image can be acquired and optionally associated with the pre-procedure ultrasound image as images for the same location in the target area.

In some embodiments, based on the guidance provided by the ultrasound system, the user is able to scan the ultrasound probe around the target area along one or more linear or angular directions from the same start position both before and after the procedure, such that respective series of ultrasound images taken of an entire three-dimensional volume before and after the procedure can be correlated by the location and posture of the ultrasound probe.

In some embodiments, if the user determines that a remedial procedure (e.g., additional ablation of the target area or nearby area) is needed based on his/her review of the post-procedure ultrasound images (e.g., relative to the pre-procedure ultrasound images), the remedial procedure can be easily performed immediately, avoiding the need for a follow-up operation on a future date.

In some embodiments, quantitative alignment information associated with acquisition of the post-procedure image data is recorded and used (e.g., as inputs, initial values, or boundary conditions, etc.) in image registration between the pre-procedure image data and the post-procedure image data, as well as with image data obtained through other imaging means.

Accordingly, in some embodiments, a system for providing guided ultrasound image acquisition includes: an ultrasound imaging system including an ultrasound probe adapted to move around an object of interest to acquire respective ultrasound image data using different probe positions; a navigation system including a navigation probe, wherein the navigation probe is adapted to be rigidly affixed to and maneuvered with the ultrasound probe within a view field of the navigation system; and a data acquisition and analysis system including one or more processors and memory, and configured to perform operations including: (1) in a first mode: acquiring first ultrasound image data while the ultrasound probe is placed in a first position; and for the first ultrasound image data, acquiring contemporaneous navigation position data of the navigation probe that is rigidly affixed to the ultrasound probe; and (2) in a second mode: generating a guidance output for assisting an operator of the ultrasound probe to physically align a current position of the ultrasound probe to the first position of the ultrasound probe associated with the first ultrasound image data.

In some embodiments, the first mode is a pre-procedure image acquisition mode and the second mode is a post-procedure image acquisition mode.

In some embodiments, the system further includes a mode-selector for selecting between the first mode and the second mode.

In some embodiments, the object of interest includes a target region of an interventional procedure within a patient's body.

In some embodiments, the first mode is used before an interventional procedure is performed on the object of interest and the second mode is used after the interventional procedure is performed on the object of interest.

In some embodiments, the navigation system further includes a reference probe adapted to be affixed in proximity to the object of interest, and to provide contemporaneous reference position data corresponding to the navigation position data acquired from the navigation probe; and the data acquisition and analysis system is further configured to: establish a dynamic reference frame based on a dynamic reference position of reference probe within the view field of the navigation system; and determine changes in the current position of the navigation probe within the dynamic reference frame.

In some embodiments, the navigation system is a magnetic navigation system including a magnetic field generator, the navigation probe is a magnetic navigation probe, the reference probe is a magnetic reference probe, and the view field of the navigation system is a magnetic field produced by the magnetic field generator of the magnetic navigation system.

In some embodiments, the magnetic field generator is physically separate from the magnetic reference probe.

In some embodiments, the magnetic field generator is physically integrated with the magnetic reference probe.

In some embodiments, the object of interest is located within a patient's body and the reference probe is affixed to a surface portion of the patient's body.

In some embodiments, the first position includes a first location and a first posture of the ultrasound probe.

In some embodiments, the guidance output includes an audio prompt for adjusting at least one of a current location and a current posture of the ultrasound probe in a respective linear or angular direction.

In some embodiments, the guidance output includes a textual prompt for adjusting at least one of a current location and a current posture of the ultrasound probe in a respective linear or angular direction.

In some embodiments, the guidance output includes graphical prompt for adjusting at least one of a current location and a current posture of the ultrasound probe in a respective linear or angular direction.

In some embodiments, the guidance output includes a first visual indicator for the first position of the ultrasound probe, and a second visual indicator for the current position of the ultrasound probe, and wherein the second visual indicator is updated in real-time as the ultrasound probe is maneuvered from the current position into the first position.

In some embodiments, the data acquisition and analysis system is further configured to perform operations including: in the second mode: determining a difference between a current position of the navigation probe relative to a previous position of the navigation probe corresponding to the first ultrasound image data; and generating the guidance output based on the determined difference.

In some embodiments, the data acquisition and analysis system is further configured to perform operations including: in the second mode: determining that the current position of the ultrasound probe has reached alignment with the first position of the ultrasound probe in accordance with predetermined alignment criteria; and acquiring second ultrasound image data from the ultrasound probe, while the ultrasound probe is in alignment with the first position of the ultrasound probe.

In some embodiments, the data acquisition and analysis system is further configured to perform operations including: in the second mode: in accordance with a determination that the current position of the ultrasound probe is in alignment with the first position of the ultrasound probe, associating the second ultrasound image data with the first ultrasound image data as image data taken using the same probe position.

In some embodiments, the data acquisition and analysis system is further configured to perform operations including: recording probe alignment information associated with acquisition of the second ultrasound image data; and utilizing the probe alignment information in image registration between the first ultrasound image and the second ultrasound image data.

Accordingly, in some embodiments, a method of providing guided ultrasound image acquisition includes: at a system including an ultrasound imaging system and a navigation system, the ultrasound imaging system including an ultrasound probe adapted to move around an object of interest to acquire respective ultrasound image data using different probe positions, and the navigation system including a navigation probe adapted to be rigidly affixed to and maneuvered with the ultrasound probe within a view field of the navigation system: (1) in a first mode: acquiring first ultrasound image data while the ultrasound probe is placed in a first position; and for the first ultrasound image data, acquiring contemporaneous navigation position data of the navigation probe that is rigidly affixed to the ultrasound probe; and (2) in a second mode: generating a guidance output for assisting an operator of the ultrasound probe to manually align a current position of the ultrasound probe to the first position of the ultrasound probe associated with the first ultrasound image data.

The details of one or more embodiments of the subject matter described in this specification are set forth in the accompanying drawings and the description below. Other features, aspects, and advantages of the subject matter will become apparent from the description, the drawings, and the claims.

### BRIEF DESCRIPTION OF THE DRAWINGS

FIG. 1 is a block diagram illustrating an operating environment of a guided ultrasound imaging system in accordance with some embodiments.

FIG. 2 is a block diagram of an exemplary data acquisition and analysis system in accordance with some embodiments.

FIGS. 3A-3B are flow charts of an exemplary method for providing guided ultrasound image acquisition in accordance with some embodiments.

Like reference numerals refer to corresponding parts throughout the drawings.

### DETAILED DESCRIPTION

At the present, during an ultrasound-guided interventional operation (e.g., a tumor ablation treatment process), ultrasound images are taken both before and after the interventional procedure is performed on the target region of a patient's anatomy. During a post-procedure review, medical personnel compares the pre-procedure and post-procedure ultrasound images of the treated area, and determines if the anticipated tumor removal objective has been sufficiently achieved, or if additional removal is needed before the operation is concluded. Sometimes, gray-scale ultrasound tissue images are used for the evaluation. Sometimes, contrast enhanced ultrasound (CEUS) images are obtained after a contrast agent is injected into the target area of the interventional procedure, both before and after the interventional procedure is performed. The review of the ultrasound images allows the medical personnel to visualize the treated area and measure the size and shape of the tumor both before the procedure and immediately after the procedure.

At present time, the measurement of tumor shape and size cannot be guaranteed to be accurate, because the pre-procedure and post-procedure ultrasound images reviewed by the medical personnel may be taken at different cross-sections using slightly different locations and postures (e.g., orientation) of the ultrasound probe. This problem is especially pronounced when the tumor area is large, and the ultrasound image cannot encompass the entire target area. Furthermore, for a large tumor of an irregular shape, different probe location and postures can produce very different resulting images that are very difficult for a viewer to visually and mentally correlate with the actual shape of the tumor. As a result, the post-procedure ultrasound images cannot be relied upon to provide an accurate assessment of whether an additional remedial procedure is needed. Thus, a method of providing consistent imaging location and probe posture before and after an interventional procedure is needed, such that a sound comparison of the pre-procedure and post-procedure ultrasound images can be made.

Although three-dimensional (3D) enhanced ultrasound imaging techniques are now available, the resulting 3D ultrasound images produced by these techniques are often displayed separately from the two-dimensional (2D) ultrasound images obtained using regular ultrasound techniques. In addition, the 3D ultrasound images are often focused on a small region of the target region, rather than the entire target region. Thus, visually and mentally correlating the 3D images and the 2D images is still a challenging task for the viewer. Sometimes, four-dimensional (4D) time sequence of 3D ultrasound images can be obtained to show dynamic changes (e.g., blood flow) within the target region. Visual and mental correlation of the pre-procedure and post-procedure 4D ultrasound images is even more challenging for the reviewer. In addition, visually correlating the ultrasound images obtained using different techniques is also difficult.

Sometimes, post-procedure assessment can be performed using other imaging equipment, such as CT/MM tomography equipment. However, imaging on such equipment is time consuming, and does not satisfy the immediacy requirement of the clinical surgery environment. For example, the CT/MRI assessment cannot be performed immediately after the performance of the interventional procedure, and before the operation is concluded. In addition, these imaging techniques also cannot provide three-dimensional volumetric quantitative comparisons of the tumor before and after the interventional procedure. Previous research focuses mostly on registration algorithm between 3D ultrasound data with CT, MRI and other 3D data, or needle guiding during the interventional procedure itself. Conventionally, most ultrasound devices allow viewing of only a single-phase 3D ultrasound image at any given time.

As described herein, an exemplary guided ultrasound imaging system includes a navigation system and an ultrasound imaging system in accordance with some embodiments. The navigation system is optionally based on a magnetic navigation system or a navigation system based on other technologies (e.g., optical camera, optical interference, triangulation based on optical or electromagnetic signal propagation to known location markers, etc.). The ultrasound imaging system is capable of perform 2D tissue imaging, 3D enhanced imaging (e.g., CEUS), or both.

The exemplary system can be used in clinical oncology intervention both before an interventional procedure is performed on a target region of a patient's anatomy, and after the interventional procedure is performed on the target region. Before the procedure, the navigation system registers location and posture information of the ultrasound probe during the ultrasound image acquisition. After the interventional procedure is performed on the target region, the exemplary system provides audio/visual guidance to the user to reposition of the ultrasound probe at the same location and/or into the same posture as before the procedure, such that a post-procedure ultrasound image may be obtained at the same probe location and/or posture as that used for a corresponding pre-procedure ultrasound image.

In some embodiments, the position information provided by the navigation system, as well as image processing techniques, is used to correlate two sets of image data acquired before and after the procedure, respectively. Once the correlation has been established between the pre-procedure and post-procedure images, measurements of the tumor can be carried out. Assessment of the tumor's shape and size, and whether the ablation region has encompassed the entire tumor area and the safety margins, can be made before the tumor removal operation is formally concluded. Optionally, if the user determines based on the assessment that the tumor has not been completely removed, or if sufficient safety margin has not been achieved, he or she may proceed to perform a remedial procedure to fill any missed areas, before the operation is formally concluded. This real-time remedial procedure helps to avoid a delayed follow-up operation to be carried out after a lengthy post-operation CT/MRI evaluation.

In addition, the quantitative alignment information (e.g., quantitative relative probe position and orientation information) associated with the pre-procedure and post-procedure image data can be used to in combination with one or other image registration techniques (e.g., rigid body translation, regression, and interactive registration, etc.) to facilitate the performance and improve the accuracy of image registration between the pre-procedure and post-procedure image data.

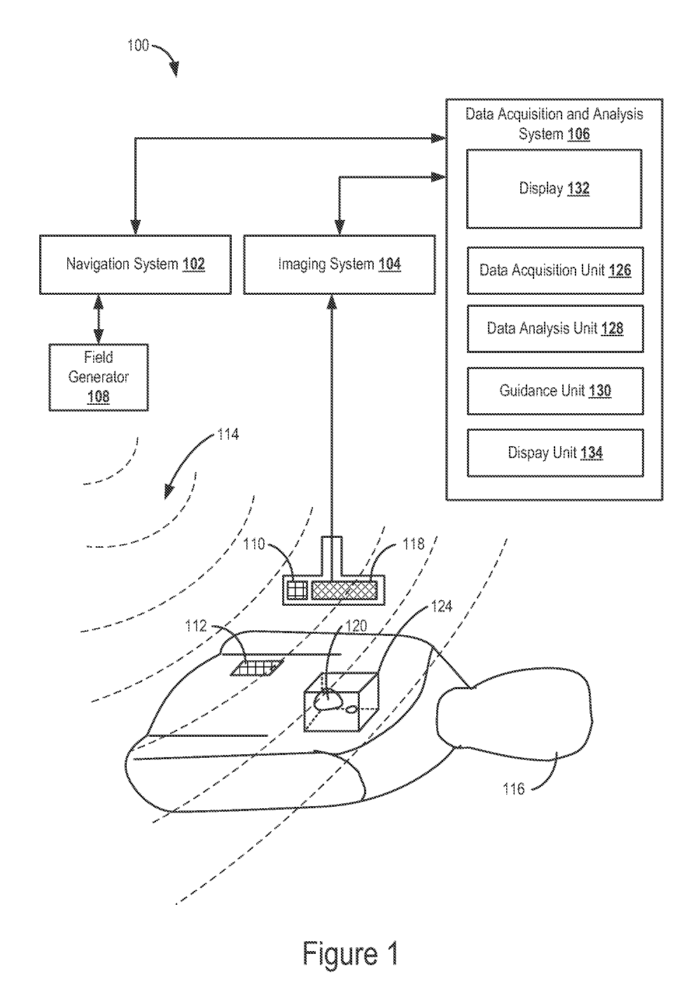

FIG. 1 is a block diagram illustrating an exemplary environment in which an exemplary guided ultrasound imaging system 100 operates to provide guided ultrasound image acquisition for immediate post-procedure evaluation and assessment. The procedure in question can be a clinical oncology treatment procedure, such as a thermal ablation of a tumor. A person skilled in the art would recognize that, other minimally invasive, interventional procedures are possible. In addition, a person skilled in the art would also recognize that, many aspects of the systems and techniques described herein are generally applicable to other applications in which acquisition and comparison of ultrasound images of the same object of interest (e.g., anatomy of an animal, equipment, a mechanical part, a terrestrial object, etc.) at different times and/or in different states are desired. Therefore, while many illustrative examples are provided herein with respect to actions occurring before and after the performance of an interventional procedure on a target area within a patient's anatomy, these actions are also generally applicable to occur before and after a change of physical state (e.g., change of content, shape, size, etc.) has occurred to an object of interest that is being imaged.

In some embodiments, the exemplary system 100 performs data registration between image data acquired before and after an interventional procedure, and displays ultrasound images based on correlated information obtained from both data sets. In some embodiments, alignment information collected at the time of acquiring the image data sets are used in improving the accuracy of the data registration.

As shown in FIG. 1, the exemplary system 100 includes a navigation system 102, an ultrasound imaging system 104, and a data acquisition and analysis system 106. In some embodiments, the data acquisition and analysis system 106 is provided by a computer, or workstation, a handheld device, or another computing device (e.g., one or more integrated circuits or chips). The navigation system 102 is coupled to the data acquisition and analysis system 106, e.g., through one or more integrated connections, wired connections, and/or wireless connections, and provides position information (e.g., location and orientation) regarding one or more probes of the navigation system 102 to the data acquisition and analysis system 106. Similarly, the ultrasound system 104 is coupled to the data acquisition and analysis system 106, e.g., through one or more integrated circuit connections, wired connections, and/or wireless connections, and provides ultrasound image data acquired through one or more probes of the ultrasound system 104 to the data acquisition and analysis system 106.

In some embodiments, the navigation system 102, the ultrasound imaging system 104, and the data acquisition and analysis system 106 are physically standalone systems that communicate with one another via one or more wired or wireless connections. In some embodiments, the ultrasound system 104 and the navigation system 102 form an integrated system having a common control unit (e.g., one or more integrated circuits or chips) that communicates with the data acquisition and analysis system (e.g., a computer, a handheld device, etc.). In some embodiments, the data acquisition and analysis system 106 is optionally integrated with a portion of the navigation system 102 and/or a portion of the ultrasound imaging system 104, such that these portions are enclosed in the same housing as the data acquisition and analysis system 106. In some embodiments, the data acquisition and analysis system 106, the navigation system 102 and the ultrasound imaging system 104 are integrated as a single device.

As shown in FIG. 1, in some embodiments, the navigation system 102 is a magnetic navigation system. In some embodiments, navigation system 102 includes a field generator 108 (e.g., a magnetic field generator), and one or more magnetic sensors (e.g., a navigation probe 110 and a reference probe 112). In operation, the field generator 108 produces a field 114 (e.g., a magnetic field) that encompasses a region large enough to enclose the lateral range of the ultrasound probe 118 over a patient's body 116. The navigation probe 110 and the reference probe 112 interact with the field 114 to produce disturbances in the field 114, which can be sensed by the field sensing elements (e.g., embedded in the field generator 108) of the navigation system 102. In some embodiments, the navigation system 102 determines the respective current locations of the navigation probe 110 and the reference probe 112 based on the changes in the field 114. In some embodiments, the navigation system 102 is further capable of determining an orientation (e.g., an angle, a heading, an orientation, etc.) of the probes 110 and 112 in a three-dimensional space. For example, in some embodiments, the probes 110 and 112 are sufficiently small, and each provides only a respective point location in the field 114. In some embodiments, the probes 110 and 112 are each of a sufficient size to accommodate multiple probe elements (e.g., magnetic coils) and are each detected in the field 114 as a line segment, a surface having a respective shape and size, or a volume having a respective shape and size.

In some embodiments, the navigation system optionally uses other navigation techniques to track the current position of the navigation probe. For example, a navigation system optionally uses optical means (e.g., an optical, CCD or infra-red camera), navigational markers (e.g., small reflective optical landmarks, EM-signal-sensing landmarks), and/or computational means (e.g., triangulation, parallax, time-difference-of-arrival, etc.) to determine the current location and/or orientation of the navigation probe.

In some embodiments, the respective location and orientation information associated with each probe of the navigation system 102 is expressed in a static reference frame, e.g., a reference frame established based on the fixed location of the field generator 108. In some embodiments, a dynamic reference system is established based on the location of the reference probe 112. The location and orientation of the navigation probe 110 is expressed in the dynamic reference system based on the relative locations and orientations between the navigation probe 110 and the reference probe 112. In some embodiments, the reference probe 112 is affixed (e.g., by an adhesive surface or adhesive tape) to a surface of the patient's body 116 near the target region 124 of the interventional procedure. Although the surface of the patient's body 116 may shift slightly during an operation, e.g., due to respiration, inadvertent movements, and changes in the underlying tissues and organs, etc., when the location and orientation of navigation probe 110 is expressed in the dynamic reference system established based on the location and orientation of the reference probe 112, the data artifacts produced by these slight movements can be effectively eliminated or reduced. In some embodiments, the reference probe 112 is sufficiently small, and serves as a single reference point (e.g., the origin) in the dynamic reference frame. In some embodiments, the reference probe 112 is of a sufficient size to accommodate multiple probe elements (e.g., magnetic coils) and provide multiple reference points establishing a 1D reference line, a 2D reference surface, or a 3D reference volume in the dynamic reference frame.

In some embodiments, the ultrasound imaging system 104 includes an ultrasound probe 118. In some embodiments, the ultrasound probe 118 includes an ultrasound transmitter that generates ultrasound waves of particular wave characteristic (e.g., frequencies, directions, etc.) and an ultrasound receiver. During operation, the ultrasound waves emitted by the ultrasound probe 118 are reflected by the objects 120 (e.g., internal tissues and structures) within the wave field (not shown) of the ultrasound probe 118. When the reflected waves are captured by the receiving elements, electric signals generated by these received waves can be used to reconstruct an image of the objects 120. In some embodiments, the ultrasound probe 118 has transmitting and receiving elements arranged in one of multiple different shaped arrays. In some embodiments, the ultrasound probe 118 emits and receives ultrasound waves in different phrases, directions, and frequencies, such that 2D, 3D, and/or 4D image data of the imaged objects may be obtained.

In some embodiments, during operation, the ultrasound probe 118 is maneuvered to different locations over the patient's body 116 near the target region 124 of the interventional procedure, and ultrasound image data of the respective regions within the view field of the ultrasound waves is acquired by the ultrasound imaging system 104. In some embodiments, 2D tissue images are obtained through the ultrasound probe 118, where each 2D image represents a respective 2D cross-section of the imaged region. In some embodiments, a contrast enhancement agent is injected into the target region, and 3D enhanced ultrasound images are obtained through the ultrasound probe 118, where each 3D image represents the imaged region at a particular time. In some embodiments, a time sequence of 3D images (i.e., 4D image data) of the same region can be obtained, to show changes of the region over time.

In some embodiments, during operation, the navigation probe 110 is rigidly attached to ultrasound probe 118, such that the navigation probe 110 and the ultrasound probe 118 can be maneuvered together (e.g., moved linearly, rotated, rocked, tilted, etc.) around the patient's body, and that the location and/or orientation of the ultrasound probe 118 can be determined from and/or approximated by the location and/or orientation of the navigation probe 110 at any give time. In some embodiments, the navigation probe 110 is rigidly attached to the ultrasound probe 119 by a clip structure, or other similar mechanical fastening means. In some embodiments, the housing of the navigation probe 110 is designed with a slot to accommodate the ultrasound probe 118. In some embodiments, the housing of the ultrasound probe 118 is designed with a slot to accommodate the navigation probe 110.

In some embodiments, the location and orientation information of the navigation probe 110 (along with the location and orientation information of the reference probe 112) is transmitted in real-time from the navigation system 102 to the data acquisition and analysis system 106, during operation of the ultrasound imaging system 104. The data acquisition and analysis system 106 determines the current location and orientation of the ultrasound probe 118 based on the current location and orientation of the navigation probe 110 relative to the reference probe 112. The data acquisition and analysis system 106 thus associates the image data acquired at any given time with the corresponding location and orientation information determined for the ultrasound probe 118. As described herein, the position of the ultrasound probe 118 optionally includes the location of the ultrasound probe 118, and/or the orientation of the ultrasound probe 118. The orientation of the ultrasound probe 118 in a three dimensional space during image acquisition is also referred to as the “posture” of the ultrasound probe 118 during image acquisition. Depending on the types of probes used, different probe postures sometimes will result in different imaging conditions, and ultimately different ultrasound images of the same imaged region.

In some embodiments, the data acquisition and analysis system 106 includes a data acquisition unit 126 that generates the instructions to control the position data acquisition from the navigation system 102, and the image data acquisition from the imaging system 104. In some embodiments, the data acquisition unit 126 correlates the position data and the image data concurrently received from the two different systems. In some embodiments, the data acquisition and analysis system 106 further includes a data analysis unit 128. In some embodiments, the data analysis unit 128 performs transformations of position data from one reference frame (e.g., a static reference frame based on the location and orientation of the field generator 108) to another reference frame (e.g., a dynamic reference frame based on the location and orientation of the reference probe 112). In some embodiments, the data analysis unit 128 further performs location and orientation determination for the image data acquired from the ultrasound probe 118. In some embodiments, if multiple imaging techniques are used, the data analysis unit 128 further performs correlation and data registration for the image data acquired based on the different imaging techniques.

In some embodiments, the data acquisition and analysis system 106 provides both a pre-procedure image acquisition mode and a post-procedure image acquisition mode for user selection, e.g., via a mode selector such as a hardware or software selection key that toggles between the two modes. In some embodiments, when the pre-procedure image acquisition has been invoked by the user, the data acquisition and analysis system 106 performs image acquisition in accordance with the movements of the ultrasound probe 118 and defers to the user (e.g., the operator of the ultrasound probe) regarding when the acquired image data is to be stored. In some embodiments, when operating in the pre-procedure image acquisition mode, the data acquisition and analysis system 106 stores image data in association with the contemporaneously acquired position information. In some embodiments, the image data acquired during the pre-procedure image acquisition is labeled as pre-procedure image data. In some embodiments, while operating in the post-procedure image acquisition mode, the data acquisition and analysis system 106 performs substantially the same functions as in the pre-procedure image acquisition mode, but the image data acquired during the post-procedure image acquisition is labeled as post-procedure image data.

In some embodiments, while operating in the post-procedure image acquisition mode, the data acquisition and analysis system 106 also actively provides guidance to the user regarding how to maneuver the ultrasound probe 118, such that image data can be acquired again at the same locations for which pre-procedure image data has been acquired and stored.

In some embodiments, while operating in the post-procedure image acquisition mode, the data acquisition and analysis system 106 also performs data registration between the pre-procedure image data and the post-procedure image data, and displays information (e.g., data, measurements, images, traces, etc.) that is generated based on both the pre-procedure image data and the post-procedure image data that have been taken with corresponding probe locations, probe postures, and/or corresponding acquisition times (e.g., time elapsed since injection of a contrast enhancement agent). More details of the post-procedure functions are provided below.

In some embodiments, the data acquisition and analysis system 106 further includes a guidance unit 130 that communicates with the data analysis unit 128 to obtain real-time location and posture information of the ultrasound probe 118. In some embodiments, when operating in the post-procedure image acquisition mode, the guidance unit 130 generates and provides guidance outputs (e.g., qualitative and/or quantitative audio/visual instructions and prompts) to assist the user to physically maneuver the ultrasound probe 118 into a position (e.g., location and/or posture) that was used to acquire another set of image data previously (e.g., before the performance of an interventional procedure).

In some embodiments, the guidance unit 130 also communicates with and controls one or more output devices (e.g., a display device 132 and/or a speaker) coupled to the data acquisition and analysis system 102, and presents the audio/visual instructions and prompts to the user (e.g., medical personnel). In some embodiments, the guidance outputs include concurrently visual indicators and/or values of a pre-procedure probe position (i.e., the target probe position) and current probe position of the ultrasound probe in a 2D or 3D coordinate system. In some embodiments, the audio/visual instructions and prompts includes a graphic representation of the target location and orientation of the ultrasound probe 118, a current location and orientation of the ultrasound probe 118, and a direction and/or angle that the ultrasound probe 118 should be moved to achieve the target location and orientation. In some embodiments, the current location and/or orientation of the ultrasound probe 118 in the graphical representation is updated in real-time, as the ultrasound probe is maneuvered by the user. In some embodiments, when alignment with the target location and/or orientation is reached in accordance with some predefined alignment criteria (e.g., linear and angular differences are less than predetermined alignment thresholds), an audio alert is generated. In some embodiments, in response to detecting that alignment with the target direction and orientation of the ultrasound probe has been achieved, the guidance unit 130 notifies the data acquisition unit 126 to acquire and store the image data in association with the current probe location and orientation. In some embodiments, the guidance unit 130 optionally also instructs the data acquisition unit 126 to store the newly acquired image data in association with other image data previously acquired using this probe location and posture. In some embodiments, additional image data may be acquired when the user scans the ultrasound probe around the target region along one or more particular linear or angular directions, if the same scanning was performed previously from the same starting probe location and posture.

In some embodiments, the data analysis unit 128 further performs data registration and correlation between the image data obtained at different times (e.g., before and after an interventional procedure), and/or using different imaging techniques (e.g., 2D tissue images, 3D enhanced ultrasound images, etc.). In some embodiments, the data analysis unit 128 performs the data registration based on the location and orientation information associated with each set of image data. In some embodiments, the data analysis unit 128 performs the data registration based on various imaging processing techniques. In some embodiments, various transformations, e.g., translation, scaling, cropping, skewing, segmentation, etc., are used to identify image data that correspond to the same objects, location, and/or time. In some embodiments, different combinations of multiple registration techniques are used to correlate the image data sets obtained at different times and/or using different imaging techniques.

In some embodiments, the data analysis unit 128 stores the quantitative alignment information (e.g., exact position data, and/or position data relative to corresponding pre-procedure probe position data) for the post-procedure imaging data, and use the quantitative alignment information in the data registration and correlation processes. For example, the alignment information can be used to provide or modify initial values, boundary values, corrections, and/or other inputs for the various data registration techniques mentioned above.

In some embodiments, once correspondence between different image data has been established by the data analysis unit 128, the correspondence is used to display images that include information obtained from both the pre-procedure image data set and the post-procedure image data set. In some embodiments, the data acquisition and analysis system 106 includes a display unit 134 that controls the concurrent display of image data that were taken using the same probe location and posture before and after the performance of the interventional procedure.

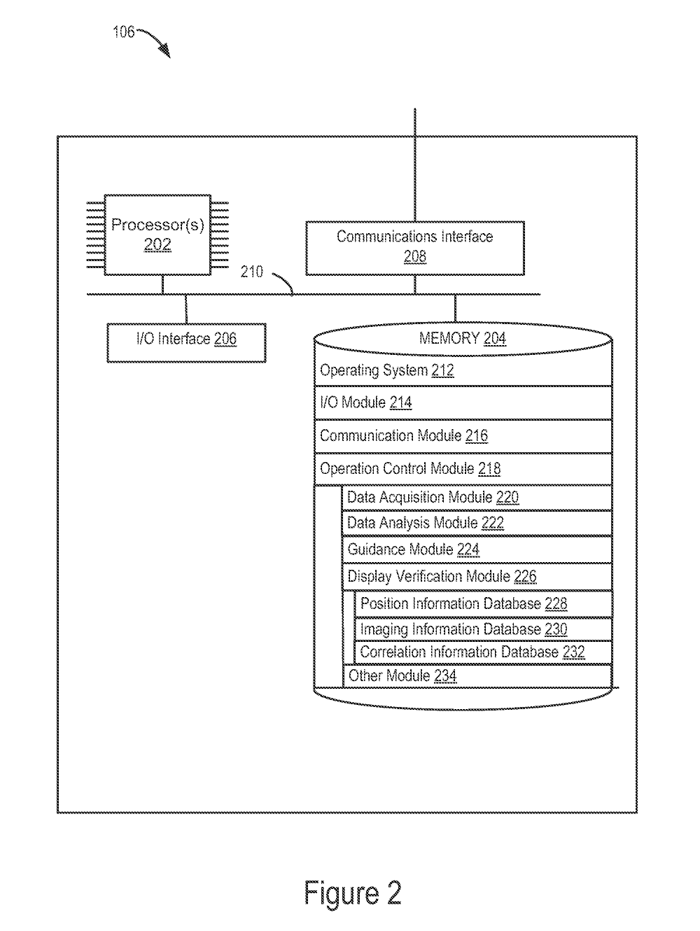

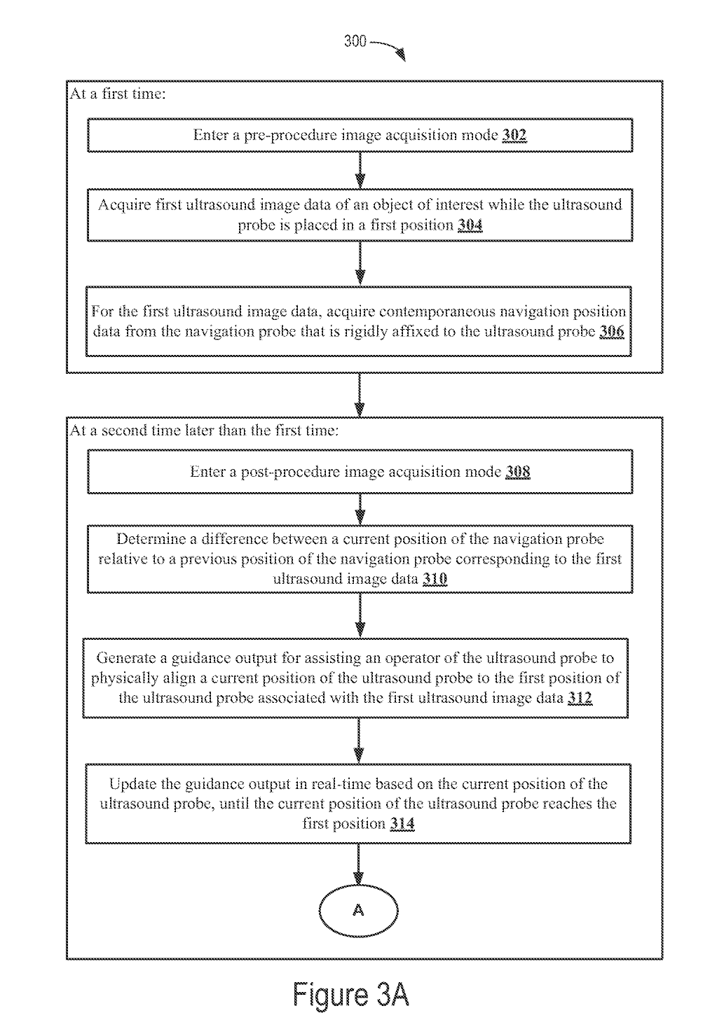

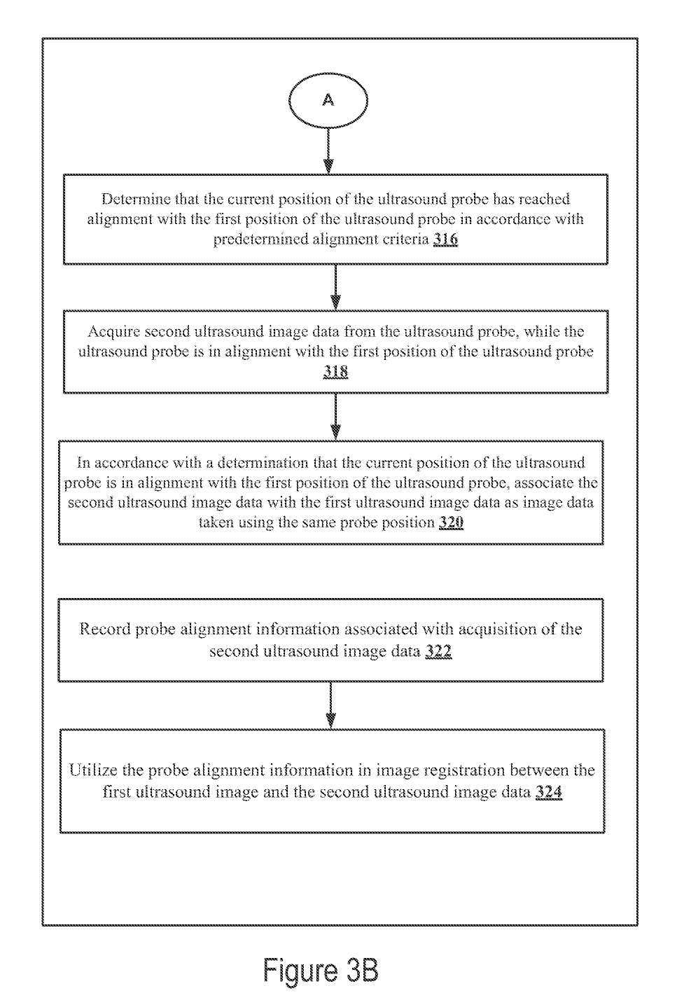

FIG. 1 provides an illustration of an exemplary guided imaging system that provides guided image acquisition for post-procedure review. The exemplary guided imaging system can be used for guided image acquisition in other situations where acquisition and comparison of ultrasound images for the same object of interest (or the same location within the object of interest) at different times are desired, and not necessary before and after an interventional procedure. Not all elements are necessary in some embodiments. In some embodiments, functions provided by some elements may be combined with functions provided by other elements. In some embodiments, one function or one element may be divided into several sub-functions and/or sub-elements. More details of the operations of the exemplary system 100 are provided below in FIGS. 2-3C, and accompany descriptions.

FIG. 2 is a block diagram of an exemplary data acquisition and analysis system 106 shown in FIG. 1, in accordance with some embodiments. As stated above, in some embodiments, the exemplary data acquisition and analysis system may be physically integrated within the same device as the ultrasound imaging system 104 and the navigation system 102. In some embodiments, different functions and/or subsystems of the data acquisition and analysis system 106 may be distributed among several physically distinct devices, e.g., between a workstation, and an integrated imaging and navigation device, or between an imaging device and a navigation device, etc.

As shown in FIG. 2, the exemplary system 106 includes one or more processing units (or “processors”) 202, memory 204, an input/output (I/O) interface 206, and a communications interface 208. These components communicate with one another over one or more communication buses or signal lines 210. In some embodiments, the memory 204, or the computer readable storage media of memory 204, stores programs, modules, instructions, and data structures including all or a subset of: an operating system 212, an I/O module 214, a communication module 216, and an operation control module 218. The one or more processors 202 are coupled to the memory 204 and operable to execute these programs, modules, and instructions, and reads/writes from/to the data structures.

In some embodiments, the processing units 202 include one or more microprocessors, such as a single core or multi-core microprocessor. In some embodiments, the processing units 202 include one or more general purpose processors. In some embodiments, the processing units 202 include one or more special purpose processors. In some embodiments, the processing units 202 include one or more personal computers, mobile devices, handheld computers, tablet computers, workstations, or one of a wide variety of hardware platforms that contain one or more processing units and run on various operating systems.

In some embodiments, the memory 204 includes high-speed random access memory, such as DRAM, SRAM, DDR RAM or other random access solid state memory devices. In some embodiments the memory 204 includes non-volatile memory, such as one or more magnetic disk storage devices, optical disk storage devices, flash memory devices, or other non-volatile solid state storage devices. In some embodiments, the memory 204 includes one or more storage devices remotely located from the processing units 202. The memory 204, or alternately the non-volatile memory device(s) within the memory 204, comprises a computer readable storage medium.

In some embodiments, the I/O interface 206 couples input/output devices, such as displays, a keyboards, touch screens, speakers, and microphones, to the I/O module 214 of the system 200. The I/O interface 206, in conjunction with the I/O module 214, receive user inputs (e.g., voice input, keyboard inputs, touch inputs, etc.) and process them accordingly. The I/O interface 206 and the user interface module 214 also present outputs (e.g., sounds, images, text, etc.) to the user according to various program instructions implemented on the system 106.

In some embodiments, the communications interface 208 includes wired communication port(s) and/or wireless transmission and reception circuitry. The wired communication port(s) receive and send communication signals via one or more wired signal lines or interfaces, e.g., twist pair, Ethernet, Universal Serial Bus (USB), FIREWIRE, etc. The wireless circuitry receives and sends RF signals and/or optical signals from/to communications networks and other communications devices. The communications module 216 facilitates communications between the system 106 and other devices (e.g., the navigation system 102 and the imaging system 104 in FIG. 1) over the communications interface 208. In some embodiments, the communications include control instructions from the data acquisition and analysis system 106 to the navigation system 102 and the imaging system 104, and location and image information from the navigation system 102 and imaging system 104 to the data acquisition and analysis system 106.

In some embodiments, the operating system 202 includes various software components and/or drivers for controlling and managing general system tasks (e.g., memory management, storage device control, power management, etc.) and facilitates communications between various hardware, firmware, and software components.

As shown in FIG. 2, the system 106 stores the operation control module 218 in the memory 204. In some embodiments, the operation control module 218 further includes the following sub-modules, or a subset or superset thereof: a data acquisition module 220, a data analysis module 222, a guidance module 224, and a display module 226. In addition, each of these sub-modules has access to one or more of the following data structures and data sources of the operation control module 218, or a subset or superset thereof: a position information database 228 containing the pre-procedure and post-procedure position information of the reference probe, the navigation probe, and the ultrasound probe; an image data database 230 containing the pre-procedure and post-procedure image data; and a correlation information database 232 which stores the location, posture, and timing correlation information for the location information and image data in the databases 228 and 230. In some embodiments, the databases 228, 230, and 232 are implemented as a single cross-linked database. In some embodiments, the operation control module 218 optionally includes one or more other modules 234 to provide other related functionalities described herein. More details on the structures, functions, and interactions of the sub-modules and data structures of the operation control module 218 are provided with respect to FIGS. 1, and 3A-3B, and accompanying descriptions.

FIGS. 3A-3B are flow diagrams of an exemplary process 300 that is implemented by an exemplary guided imaging system (e.g., the exemplary system 100, or the data acquisition and analysis system 106 of FIG. 1).

As discussed above with respective to the exemplary system 100 shown in FIG. 1, in some embodiments, the guided imaging system 100 includes an ultrasound imaging system (e.g., imaging system 104 in FIG. 1) and a navigation system (e.g., navigation system 102 in FIG. 1). The ultrasound imaging system includes an ultrasound probe (e.g., ultrasound probe 118) adapted to move around an object of interest (e.g., target region 124 of an interventional procedure in FIG. 1) to acquire respective ultrasound image data of the object of interest using different probe positions. In some embodiments, the navigation system includes a navigation probe and is configured to track the current position of the navigation probe within a view field of the navigation system. In some embodiments, the view field of the navigation system is a region of space in which position of the navigation probe can be determined through a monitoring mechanism of the navigation system. In some embodiments, the navigation system is a magnetic navigation system, and the view field of the navigation system is a magnetic field generated by a magnetic field generator of the navigation system. The navigation system optionally senses the position of the navigation probe based on field disturbances caused by the navigation probe. In some embodiments, the navigation system is an optical navigation system, and a view field of the navigation system is the combined view fields of one or more optical, infra-red, and/or CCD cameras directed at the object of interest. The navigation system optionally senses the position of the navigation probe based on projections or images of the navigation probe formed in the cameras. In some embodiments, the navigation system includes two or more signal-sensing landmarks (e.g., laser-beam sensing, or other EM-signal sensing) with known positions, and the view field of the navigation system is the combined signal-sensing range of the signal-sensing landmarks. The navigation system optionally determines (e.g., based on triangulation or other geometric or mathematic methods) the position of the navigation probe based on the direction and/or timing of the optical or EM signals emitted by the navigation probe and received by the different signal-sensing landmarks. Navigation systems based on other techniques and components are possible.

In some embodiments, the navigation probe is adapted to be rigidly affixed to and maneuvered with the ultrasound probe (e.g., ultrasound probe 118 in FIG. 1) within the view field of the navigation system. In some embodiments, the navigation system further includes a reference probe adapted to be affixed in proximity to the object of interest, and to provide contemporaneous reference position data corresponding to the navigation position data acquired from the navigation probe.

In some embodiments, the navigation system is a magnetic navigation system that includes a magnetic field generator (e.g., field generator 108 in FIG. 1), and a magnetic navigation probe (e.g., navigation probe 110 in FIG. 1) adapted to be rigidly affixed to and maneuvered with the ultrasound probe (e.g., ultrasound probe 118 in FIG. 1) within a magnetic field (e.g., field 114 in FIG. 1) produced by the magnetic field generator. In some embodiments, the magnetic navigation system further includes a magnetic reference probe (e.g., reference probe 112 in FIG. 1) adapted to be affixed to a portion of an anatomy that is located in proximity to the target region (e.g., target region 124 in FIG. 1), and to provide contemporaneous reference position data corresponding to the navigation position data acquired from the magnetic reference probe (e.g., the navigation probe 110 in FIG. 1).

In some embodiments, the ultrasound imaging system is connected to the ultrasound probe, and transmits and receives specific ultrasonic waveform to and from the ultrasound probe. The ultrasound imaging system processes the received waveforms to generate image data for the tissue within the target region. In some embodiments, the magnetic navigation system includes magnetic field transmitter and signal receiver modules that are connected to the reference probe and the navigation probe wirelessly or via data lines. The reference probe serves to provide a means to determine the current body position of the patient, and the navigation probe serves to provide a means to determine the current position of the ultrasound probe. Specifically, the spatial coordinates of the current position and orientation of the positioning devices (e.g., the reference probe and the navigation probe) can be denoted as a set of coordinates (x, y, z, a, b, c) in a static reference frame (e.g., a reference frame based on the magnetic field 114). The first three coordinates (e.g., x, y, z) of the current position of the positioning device are location coordinates relative to the static reference frame (e.g., the magnetic field reference frame). The latter three coordinates (e.g., a, b, c) for the current position of the positioning device are posture or rotation coordinates relative to the static reference frame (e.g., the magnetic field reference frame).

In some embodiments, the reference probe (e.g., reference probe 112) is placed within the view field (e.g., field 114) of the navigation system at a location on a surface of the patient's body (e.g., patent's body 116 in FIG. 1). The reference probe thus provides real-time information regarding the current position of the patient's body. Based on the position information received from the reference probe, the current location and the current orientation of the patient's body near the target region can be determined in real-time. In some embodiments, the reference probe can be affixed to the patient's body used double-sided adhesive tape, bandages and other fixed attachment. In normal operation, the patient is advised to keep completely still during the entire image scanning process. However, small inadvertent movements or unavoidable movements (e.g., due to body tremors or respiratory movements) can be tolerated, as discussed in more detail below.

In some embodiments, the navigation probe is placed within the view field (e.g., field 114) of the navigation system, and returns in real-time the current position (e.g., the location and orientation) of the navigation probe. In some embodiments, when in use, the navigation probe is rigidly affixed to the ultrasound probe, such that the real-time position information received from the navigation probe can be used to determine the real-time current position (e.g., current location and orientation) of the ultrasound probe. In some embodiments, a specially designed slot can be used to fit the two probes in fixed relative position during operation.

In some embodiments, as the ultrasound imaging system transmits image data to the data acquisition and analysis system of the guided imaging system, the navigation system transits concurrent real-time position information of the reference probe and the navigation probe to the data acquisition and analysis system. For example, the reference probe position is represented by a first set of coordinates R1=(x1, y1, z1, a1, b1, c1) and the navigation probe position is represented by a second set of coordinates R2=(x2, y2, z2, a2, b2, c2). Both sets of coordinates R1 and R2 are expressed in the static reference system of the magnetic field of the navigation system. In some embodiments, acquisition time information is associated with the image data received from the ultrasound probe and the position information received from the reference and navigation probes.

In some embodiments, the data acquisition and analysis system (or a sub-module thereof) establishes a dynamic reference frame based on a dynamic reference position (e.g., R1) of the reference probe within the view field of the navigation system (e.g., the magnetic field produced by the magnetic field generator of the magnetic navigation system). In some embodiments, the data acquisition and analysis system (or a sub-module thereof) determines the difference between the current position (e.g., a post-procedure position) of the navigation probe relative to the previous position (e.g., a pre-procedure position) of the navigation probe within the dynamic reference frame. For example, the current position of the navigation probe can be expressed in the dynamic reference frame of the reference probe as R3_{T2}=(R2_{T2}−R1_{T2}), while the previous position of the navigation probe can be expressed in the dynamic reference frame of the reference probe as R3_{T1}=(R2_{T1}−R1_{T1}), wherein T₂ is a data acquisition time after the interventional procedure, and T₁ is a data acquisition time before the interventional procedure.

In some embodiments, a coordinate conversion table can be used to transform the position coordinates of the navigation probe in the static reference frame of the view field, to the position coordinates of the navigation probe in the dynamic reference frame of the reference probe. In addition, based on the position coordinates of the navigation probe in the dynamic reference frame, the position coordinates of the ultrasound probe are determined.

In some embodiments, it is advantageous to include the reference probe in the navigation system, such that position coordinates of the ultrasound probe at different times can be expressed in a consistent manner in the same reference frame, irrespective to the movement of the patient's body during the imaging process.

As described in this specification, the ultrasound imaging system is configured to obtain multiple groups of ultrasound image data using different probe positions. By correlating the contemporaneously received probe position information and image data, post processing of the position information and image data can be carried out to display ultrasound images in a manner that is intuitive and meaningful.

In some embodiments, the ultrasound imaging system includes an ultrasound probe capable of obtaining 2D ultrasound image data, 3D ultrasound image data, and/or 4D ultrasound image data. In some embodiments, the ultrasound imaging system includes one or more ultrasound probes, each being rigidly affixed to a respective navigation probe. In some embodiments, the different ultrasound probes can be used at different times.

As shown in the exemplary process 300 in FIG. 3A, at a first time (e.g., before an interventional procedure is performed on a target region of the patient's anatomy), an operator provides an input (e.g., pressing a mode selection key, or power on the system) to invoke a pre-procedure image acquisition mode of the guided ultrasound imaging system. In response to the user input, the guided ultrasound imaging system enters (302) a pre-procedure image acquisition mode. While operating in the pre-procedure image acquisition mode, the system acquires (304) first ultrasound image data of an object of interest (e.g., the target region of the patient's anatomy) while the ultrasound probe is placed in a first position (e.g., a first location and/or a first orientation). For the first ultrasound image data, the system also acquires (306) contemporaneous navigation position data from the navigation probe (e.g., the magnetic navigation probe) that is rigidly affixed to the ultrasound probe.

In some embodiments, depending on the type of ultrasound probe used, the first ultrasound image data includes 2D tissue image data, 3D volume image data, 3D contrast enhanced image data, and/or 4D time sequence of volume image data, etc. Although the first image data may be of different imaging parameters, such as imaging depth, zoom level, acquisition time, pulse repetition frequency, contrast, etc., the first image data is acquired using a first probe position. In addition, although multiple ultrasound images can be generated based on the first image data, each of the multiple ultrasound images are also associated with the same first probe position.

In some embodiments, the first image data is image data acquired while the ultrasound probe is in a starting position. In some embodiments, after the operator moves the ultrasound probe to a start position, the operator optionally scans the ultrasound probe along one or more linear and/or angular directions to acquire more image data around the object of interest (e.g., the target region of the interventional procedure). For example, the operator optionally maintains the orientation of the ultrasound probe, and scans a planar rectangular area over the target region. In some embodiments, the operator optionally rotates the ultrasound probe and scans a cone of 135 degree angle, while keeping the linear location of the ultrasound probe unchanged. In some embodiments, the operator optionally vary the scanning depth or scanning wavelength of the ultrasound probe to obtain images at different body depth, or obtain images of objects having different tissue characteristics.

In some embodiments, based on the real-time position information provided by the magnetic navigation system, the guided imaging system stores all of the subsequently acquired image data with their corresponding contemporaneous position information. In some embodiments, the image data sets obtained during each scan are optionally stored in sequence according to the order by which they have been obtained. Scanning object of interest (e.g., the target region of the interventional procedure) using different probe positions and/or imaging conditions, allow more comprehensive image data to be acquired for the object of interest. In some embodiments, the captured image data can include ordinary tissue image data, and enhanced ultrasound image data, or both. In some embodiments, ordinary tissue images and enhanced ultrasound images can be obtained simultaneously using the same ultrasound probe, and the points or pixels within tissue images and enhanced images have one-to-one correspondence.

As described above, the respective position coordinates of the navigation probe and the ultrasound probe can be expressed in the dynamic reference system based on the position of reference probe within the view field of the navigation system (e.g., the magnetic field produced by a magnetic field generator of a magnetic navigation system). For each set of image data collected using a particular ultrasound probe position, the position coordinates can be expressed as P3=(x3, y3, z3, a3, b3, c3)=P1−P2, where P1 is the position of the ultrasound probe that has been determined in the static reference frame of the view field, while P2 is the position of the ultrasound probe when the navigation probe is placed at the same location R2 of the reference probe in the static reference frame of the view field. In some embodiments, when the reference probe and the navigation probe are small, and the distance between the ultrasound probe and the navigation probe is negligible, the position (e.g., location and/or orientation) of the ultrasound probe can be approximated by the position of navigation probe.

In some embodiments, when a magnetic navigation system is used, for easy of computation, the field generator of the navigation system is optionally integrated with the reference probe and affixed to the surface of the patient's body. Thus, the static reference system based on the magnetic field, and the dynamic reference system based on the position of the reference probe merge into the same reference system. As a result, the position coordinates for the navigation probe can be obtained directly from the navigation system, and no conversion of reference systems is needed. In addition, the position information of the reference probe is no longer necessary. In some embodiments, the magnetic field generator is physically separate from the magnetic reference probe. In some embodiments, the magnetic field generator is physically integrated with the magnetic reference probe.

In some embodiments, after sufficient amount of pre-procedure ultrasound image data have been acquired, the medical personnel can proceed to perform the interventional procedure on the target region of the patient's anatomy as planned. For example, in some instances, thermal ablation of one or more tumors within the target region is performed using an ablation needle. In some embodiments, the interventional procedure is guided by the previously obtained ultrasound images, or real-time ultrasound images.

After the interventional procedure has been completed according to plan, or after a suitable stop point of the procedure is reached, the medical personnel can stop the procedure, and performs post-procedure evaluation of the target region to determine if additional remedial procedure is needed in the target region. In some embodiments, the operator provides another input to invoke the post-procedure image acquisition mode of the guided imaging system, e.g., by pressing a mode selection key or a mode toggle button.

In some embodiments, as shown in FIG. 3A, at a second time later than the first time (e.g., after the interventional procedure or a suitable stop point): in response to the user input, the guided ultrasound imaging system enters (308) the post-procedure image acquisition mode. During the post-procedure image acquisition mode, the guided imaging system optionally determines (310) a difference between a current position of the magnetic navigation probe relative to a previous position of the magnetic navigation probe corresponding to the first ultrasound image data. In some embodiments, the guided imaging system generates (312) a guidance output for assisting an operator of the ultrasound probe to physically align (e.g., by hand or by another mechanical or electronic means) a current position of the ultrasound probe to the first position of the ultrasound probe associated with the first ultrasound image data. In some embodiments, the guided imaging system generates the guidance output based on the determined difference. In some embodiments, the guided imaging system updates (314) the guidance output in real-time based on the current position of the ultrasound probe, until the current position of the ultrasound probe reaches the first position.

For example, in some embodiments, the operator places the ultrasound probe in a location at or near a start location of a particular scan previously performed before the interventional procedure, and holds the ultrasound probe in a posture that is the same or similar to the start posture of the particular scan previously performed. The guided imaging system determines the current ultrasound probe position based on the current position of the navigation probe in the dynamic reference frame. The guided imaging system further obtains the stored position of the ultrasound probe previously used to obtain a set of pre-procedure ultrasound image data, where the stored position is expressed in the dynamic reference system based on the previous position of the reference probe at the time of pre-procedure image acquisition. The guided imaging system determines the difference between the two positions of the ultrasound probe, and generates a guidance output to assist the operator to move the ultrasound probe in a way such that the ultrasound probe can be placed into the same position as that used for the pre-procedure image acquisition. In some embodiments, as the operator continues to move the ultrasound probe, additional guidance outputs are generated and presented to the user in real-time, such that the guidance is always appropriate for the current location and posture of the ultrasound probe.

In some embodiments, the guided imaging system generates an audio prompt for adjusting at least one of a current location and a current posture of the ultrasound probe in a respective linear or angular direction. For example, the audio prompt is optionally an audio instruction, such as “move the ultrasound probe to the left by 0.5 cm”, “rotate the ultrasound probe clockwise by 5 degrees”, “tilt the ultrasound probe forward by 4 degrees,” “pan the ultrasound probe to the left by 10 degrees”, etc. In some embodiments, the guided imaging system generates a textual prompt for adjusting at least one of a current location and a current posture of the ultrasound probe in a respective linear or angular direction. For example, in some embodiments, the audio prompts given above are optionally displayed as textual prompts on a display device of the guided imaging system, contemporaneously with the ultrasound images acquired using the current position of the ultrasound probe. In some embodiments, the audio prompt optionally only specifies a particular movement and direction (e.g., tilt, pan, rotate, shift, forward, backward, clockwise, counterclockwise, left, right, etc.), while the textual prompt provides the exact amount of movement needed. In some embodiments, the audio and textual prompts are updated in real-time to reflect the changes in the probe position that the operator has already caused in response to the earlier prompts.

In some embodiments, the guided imaging system generates a graphical prompt for adjusting at least one of a current location and a current posture of the ultrasound probe in a respective linear or angular direction. For example, an outline or image of the ultrasound probe is optionally displayed on a display device of the guided imaging system, and an animation is played to indicate the desired movement of the ultrasound probe to bring the ultrasound probe into position. In some embodiments, the animation is updated in real-time to reflect the changes in the probe position that the operator has already caused in response to the graphical prompt.

In some embodiments, the guided imaging system displays, on a display device, a first visual indicator (e.g., a graphic location marker and/or coordinate values) for the first position of the ultrasound probe, and a second visual indicator (e.g., a graphic location marker and/or coordinate values) for the current position of the ultrasound probe, and updates the second visual indicator in real-time as the ultrasound probe is maneuvered from the current position into the first position.

In some embodiments, as shown in FIG. 3B, after the interventional procedure is performed on the target region, e.g., after the operator has correctly maneuvered the ultrasound probe according to the guidance outputs, the guided imaging system determines (316) that the current position of the ultrasound probe has reached alignment with the first position of the ultrasound probe in accordance with predetermined alignment criteria (e.g., with an alignment error less than a threshold value). In some embodiments, the guided imaging system acquires (318) second ultrasound image data from the ultrasound probe, while the ultrasound probe is in alignment with the first position of the ultrasound probe. In some embodiments, in accordance with a determination that the current position of the ultrasound probe is in alignment with the first position of the ultrasound probe, the guided imaging system associates (320) the second ultrasound image data with the first ultrasound image data as image data taken using the same probe position. In some embodiments, the second image data are of the same types as the first image data. In some embodiments, the second image data includes more or fewer types of data than the first image data.

In some embodiments, once the alignment of the start position has been reached under the guidance of the guided imaging system, the guided imaging system further provides additional audio/visual guidance prompts to the operator. The additional guidance prompts instructs and assists the operator to perform the same scans (e.g., scans in one or more directions and angles, depth, frequencies, etc.) as those performed during the pre-procedure scan time. For example, once the ultrasound probe has reached a starting position for a scan performed before the interventional procedure, the guidance prompt optionally includes an instruction to guide the scan, e.g., “slowly move the probe back and forth to scan a 20 cm×20 cm rectangular area” or “slowly tilt the probe backward to scan a 90 degree angle,” or “hold the probe steady for 10 seconds”, “gradually increasing scanning depth from 1 cm to 10 cm” etc. In some embodiments, as the operator continues to maneuver the ultrasound probe and/or imaging conditions (e.g., frequency, depth, etc.) in accordance with the guidance instruction, the audio/visual guidance prompts optionally shows the progress of the scan based on the current position of the ultrasound probe, and all positions required to complete the scan. In some embodiments, the acquisition time, probe position, and/or imaging conditions are recorded and stored with the additional image data obtained during the scan.

In some embodiments, the image data acquired during the post-procedure scans are automatically correlated with the image data acquired during the pre-procedure scans. The correlation or data registration between the multiple sets of image data are optionally based on the respective position information associated the different sets of pre-procedure and post-procedure ultrasound image data. In some embodiments, the correlation or data registration between the multiple sets of image data are further optionally based on the respective time information and other imaging condition information associated the different sets of pre-procedure and post-procedure ultrasound image data. In some embodiments, once the different image data sets are correlated, the guided imaging system is able to generate one or more ultrasound images from each data set, identify the correspond data set(s), and generate one or more corresponding ultrasound images from the corresponding data sets. In some embodiments, the corresponding images from the different image data sets correspond to each other in at least one of probe location, probe posture, image location, imaging time, imaging depth, and imaging frequency, etc.

In some embodiments, once the different image data sets are correlated, and pixels within the different images generated from the different image data sets are registered with one another, the guided imaging system optionally presents the corresponding ultrasound images to the user for concurrent review.

In some embodiments, at least some image acquired before and after the procedure is not necessarily guided by the guided imaging system, and may be entirely decided by the medical personnel. However, because the navigation system provides real-time position information (e.g., real-time probe location and posture information) during both the pre-procedure and the post-procedure image acquisition, all image data that has been acquired can be associated with corresponding probe positions. In addition, the reference probe is used to establish a dynamic reference frame that is robust enough in light of inadvertent and/or unavoidable movements of the patient's body, such that when the probe positions of the ultrasound probe are expressed in the dynamic reference frame established based on the reference probe, the imaging positions can be consistently compared and correlated. Thus, for each pre-procedure image frame, a corresponding post-procedure image frame can be identified and displayed concurrently. In addition, when concurrently displaying pre-procedure and post-procedure image frames in the same display, other image processing techniques can be used, such that the concurrently displayed images are of the same scale, and location, depth, and/or other imaging conditions.

In some embodiments, the mapping between a pre-procedure image and a post-procedure image includes a rigid body transformation (e.g., translation and rotation) M0=(x0, y0, z2, a0, b0, c0), where the transformation M0 is determined based on the difference between the ultrasound probe positions in the dynamic reference system established based on the position of the reference probe.

In some embodiments, when a pre-procedure image and a post-procedure image are displayed on the same screen, one image may be obtained from a particular pre-procedure image data set, and the other image may be obtained from a particular post-procedure image data set, where the two images correspond to the same imaging location (pixel-by-pixel) in the target region according to the correspondence between the probe positions of the pre-procedure and the post-procedure data sets.

In some embodiments, due to the difference in imaging conditions, and movements of the patients' skin under the reference probe, there may be some remaining discrepancies in the position information stored by the guided imaging system. In some embodiments, image processing techniques can be used to further improve the alignment of the pre-procedure and post-procedure image data sets. In some embodiments, the stored position information can be used as initial values or constraints for the data registration computation.

In some embodiments, data registration can be based on the tissue image data, the contrast enhanced ultrasound image data, or both. In some embodiments, the automatic image registration algorithm is based on image similarity and image mapping considerations. In some embodiments, different mapping methods include rigid body transformation (e.g., rotation and translation), projection transformation (e.g., scaling, rotation, and translation), and non-linear transformation (e.g., using different mappings for different parts of the images). As a person skilled in the art would recognize, other data registration methods are possible.

In some embodiments, if two sets of image data are collected under the same depth, the pixels in the images are of the same scale. As such, the registration algorithm can be confined to rigid body transformation, which includes rotation and translation. The rigid body transformation can be expressed as M0=(x0, y0, z2, a0, b0, c0), or a formula (1) in the form of matrix A. If the collection depths are different, a bilinear difference algorithm can be used to scale the data into the same size, and followed by a rigid body transformation to achieve the registration. For example, suppose that, in a CEUS image, a pixel Xᵢ has a brightness f(Xi), and in another CEUS image, a pixel Yⱼ has a brightness f(Yj), then a mapping between the two images can be expressed by:

$${X}_{i} = {AY}_{j} , {X}_{i} = [ \begin{matrix}{x}_{1}^{i} \\ {x}_{2}^{i} \\ {x}_{3}^{i} \\ 1\end{matrix} ] , {Y}_{j} = [ \begin{matrix}{y}_{1}^{j} \\ {y}_{2}^{j} \\ {y}_{3}^{j} \\ 1\end{matrix} ] , A = [ \begin{matrix}{a}_{11} & {a}_{12} & {a}_{13} & {T}_{1} \\ {a}_{21} & {a}_{22} & {a}_{23} & {T}_{2} \\ {a}_{31} & {a}_{32} & {a}_{33} & {T}_{3} \\ 0 & 0 & 0 & 1\end{matrix} ] \qquad ( 1 )$$

At the same time, a similarity function can be defined as

$$E = {\sum }_{i = 1 … N} \vert f ( {X}_{i} ) - g ( {AX}_{i} ) \vert ,$$

which is used in a smallest absolute difference (SAD) method. Similarly, algorithms such as least square difference (SSD), maximum cross-correlation (C-C), and improved smallest absolute difference (SAD) based on the characteristic Rayleigh distribution of ultrasonic noise, etc., can be used for the data registration process. In some embodiments, in addition to f(Xᵢ), and f(Yᵢ), other functions based on the regional size gradient, and regional brightness gradient can also be defined. As a person skilled in the art would recognize, other automatic registration algorithms are possible.

In some embodiments, in addition to automatic data registration processes, interactive registration methods are also be provided. In some embodiments, a number of (e.g., four or more) corresponding points are identified by the user in the images to be registered, and based on these corresponding points, an automatic registration algorithm can be used to perform data registration of the images using a least-square fit method. In some embodiments, two corresponding cross-sections can be identified by the user, and based on the corresponding cross-sections, correspondence between two sets of volume data may be determined.

In some embodiments, the quantitative alignment information (e.g., quantitative relative probe position and orientation information) associated with the pre-procedure and post-procedure image data can be used to in combination with one or other image registration techniques (e.g., rigid body translation, regression, and interactive registration, etc.) to facilitate the performance and improve the accuracy of image registration between the pre-procedure and post-procedure image data. For example, in some embodiments, the guided imaging system records (322) probe alignment information (e.g., qualitative and/or quantitative errors in alignment, exact position values, and/or relative position values) associated with acquisition of the second ultrasound image data, and utilizes (324) the probe alignment information in image registration between the first ultrasound image and the second ultrasound image data.

The above exemplary process is merely provided to illustrate the principles of the techniques described herein. Not all steps need to be performed in a particular embodiment. Unless specifically stated, the order of the steps may be different in various embodiments.

The foregoing description, for purpose of explanation, has been described with reference to specific embodiments. However, the illustrative discussions above are not intended to be exhaustive or to limit the invention to the precise forms disclosed. Many modifications and variations are possible in view of the above teachings. The embodiments were chosen and described in order to best explain the principles of the invention and its practical applications, to thereby enable others skilled in the art to best utilize the invention and various embodiments with various modifications as are suited to the particular use contemplated.

1. A system for providing guided ultrasound image acquisition, comprising:
an ultrasound imaging system comprising an ultrasound probe adapted to move around an object of interest to acquire respective ultrasound image data using different probe positions;
a navigation system comprising a navigation probe, wherein the navigation probe is adapted to be rigidly affixed to and maneuvered with the ultrasound probe within a view field of the navigation system; and
a data acquisition and analysis system comprising one or more processors and memory, and configured to perform operations comprising:
in a first mode:
acquiring first ultrasound image data while the ultrasound probe is placed in a first position; and
for the first ultrasound image data, acquiring contemporaneous navigation position data of the navigation probe that is rigidly affixed to the ultrasound probe;
in a second mode:
generating a guidance output for assisting an operator of the ultrasound probe to physically align a current position of the ultrasound probe to the first position of the ultrasound probe associated with the first ultrasound image data.

2. The system of claim 1, wherein the first mode is a pre-procedure image acquisition mode and the second mode is a post-procedure image acquisition mode.

3. The system of claim 1, wherein the system further comprises a mode-selector for selecting between the first mode and the second mode.

4. The system of claim 1, wherein the object of interest comprises a target region of an interventional procedure within a patient's body.

5. The method of claim 1, wherein the first mode is used before an interventional procedure is performed on the object of interest and the second mode is used after the interventional procedure is performed on the object of interest.

6. The system of claim 1, wherein:
the navigation system further comprises a reference probe adapted to be affixed in proximity to the object of interest, and to provide contemporaneous reference position data corresponding to the navigation position data acquired from the navigation probe; and
the data acquisition and analysis system is further configured to:
establish a dynamic reference frame based on a dynamic reference position of reference probe within the view field of the navigation system; and
determine changes in the current position of the navigation probe within the dynamic reference frame.

7. The system of claim 6, wherein the navigation system is a magnetic navigation system comprising a magnetic field generator, the navigation probe is a magnetic navigation probe, the reference probe is a magnetic reference probe, and the view field of the navigation system is a magnetic field produced by the magnetic field generator of the magnetic navigation system.

8. The system of claim 7, wherein the magnetic field generator is physically separate from the magnetic reference probe.

9. The system of claim 7, wherein the magnetic field generator is physically integrated with the magnetic reference probe.

10. The system of claim 6, wherein the object of interest is located within a patient's body and the reference probe is affixed to a surface portion of the patient's body.

11. The system of claim 1, wherein the first position includes a first location and a first posture of the ultrasound probe.

12. The system of claim 1, wherein the guidance output includes an audio prompt for adjusting at least one of a current location and a current posture of the ultrasound probe in a respective linear or angular direction.

13. The system of claim 1, wherein the guidance output includes a textual prompt for adjusting at least one of a current location and a current posture of the ultrasound probe in a respective linear or angular direction.

14. The system of claim 1, wherein the guidance output includes graphical prompt for adjusting at least one of a current location and a current posture of the ultrasound probe in a respective linear or angular direction.

15. The system of claim 1, wherein the guidance output includes a first visual indicator for the first position of the ultrasound probe, and a second visual indicator for the current position of the ultrasound probe, and wherein the second visual indicator is updated in real-time as the ultrasound probe is maneuvered from the current position into the first position.

16. The system of claim 1, wherein the data acquisition and analysis system is further configured to perform operations comprising:
in the second mode:
determining a difference between a current position of the navigation probe relative to a previous position of the navigation probe corresponding to the first ultrasound image data; and
generating the guidance output based on the determined difference.

17. The system of claim 1, wherein the data acquisition and analysis system is further configured to perform operations comprising:
in the second mode:
determining that the current position of the ultrasound probe has reached alignment with the first position of the ultrasound probe in accordance with predetermined alignment criteria; and
acquiring second ultrasound image data from the ultrasound probe, while the ultrasound probe is in alignment with the first position of the ultrasound probe.

18. The system of claim 17, wherein the data acquisition and analysis system is further configured to perform operations comprising:
in the second mode:
in accordance with a determination that the current position of the ultrasound probe is in alignment with the first position of the ultrasound probe, associating the second ultrasound image data with the first ultrasound image data as image data taken using the same probe position.

19. The system of claim 18, wherein the data acquisition and analysis system is further configured to perform operations comprising:
recording probe alignment information associated with acquisition of the second ultrasound image data; and
utilizing the probe alignment information in image registration between the first ultrasound image and the second ultrasound image data.

20. A method of providing guided ultrasound image acquisition, comprising:
at a system comprising an ultrasound imaging system and a navigation system, the ultrasound imaging system comprising an ultrasound probe adapted to move around an object of interest to acquire respective ultrasound image data using different probe positions, and the navigation system comprising a navigation probe adapted to be rigidly affixed to and maneuvered with the ultrasound probe within a view field of the navigation system:
in a first mode:
acquiring first ultrasound image data while the ultrasound probe is placed in a first position; and
for the first ultrasound image data, acquiring contemporaneous navigation position data of the navigation probe that is rigidly affixed to the ultrasound probe;
in a second mode:
generating a guidance output for assisting an operator of the ultrasound probe to manually align a current position of the ultrasound probe to the first position of the ultrasound probe associated with the first ultrasound image data.

21. The method of claim 20, wherein the first mode is a pre-procedure image acquisition mode and the second mode is a post-procedure image acquisition mode.

22. The method of claim 20, further comprising:
selecting the first mode before performing a procedure for changing a physical state of the object of interest; and
selecting the second mode after performing the procedure for changing the physical state of the object of interest.

23. The method of claim 20, wherein the object of interest is a target region of an interventional procedure within a patient's body.

24. The method of claim 20, wherein the first mode is used before an interventional procedure is performed on the object of interest and the second mode is used after the interventional procedure is performed on the object of interest.

25. The method of claim 20, wherein:
the navigation system further comprises a reference probe adapted to be affixed in proximity to the object of interest, and to provide contemporaneous reference position data corresponding to the navigation position data acquired from the navigation probe; and
the method further comprises:
establishing a dynamic reference frame based on a dynamic reference position of reference probe within the view field of the navigation system; and
determining changes in the current position of the navigation probe within the dynamic reference frame.

26. The method of claim 25, wherein the navigation system is a magnetic navigation system comprising a magnetic field generator, the navigation probe is a magnetic navigation probe, the reference probe is a magnetic reference probe, and the view field of the navigation system is a magnetic field produced by the magnetic field generator of the navigation system.

27. The method of claim 26, wherein the magnetic field generator is physically separate from the magnetic reference probe.

28. The method of claim 26, wherein the magnetic field generator is physically integrated with the magnetic reference probe.

29. The method of claim 26, wherein the object of interest is located within a patient's body and the reference probe is affixed to a surface portion of the patient's body.

30. The method of claim 20, wherein the first position includes a first location and a first posture of the ultrasound probe.

31. The method of claim 20, wherein generating a guidance output further comprises:
generating an audio prompt for adjusting at least one of a current location and a current posture of the ultrasound probe in a respective linear or angular direction.

32. The method of claim 20, wherein generating a guidance output further comprises:
generating a textual prompt for adjusting at least one of a current location and a current posture of the ultrasound probe in a respective linear or angular direction.

33. The method of claim 20, wherein generating a guidance output further comprises:
generating a graphical prompt for adjusting at least one of a current location and a current posture of the ultrasound probe in a respective linear or angular direction.

34. The method of claim 20, wherein generating a guidance output further comprises:
displaying, on a display device, a first visual indicator for the first position of the ultrasound probe, and a second visual indicator for the current position of the ultrasound probe; and
updating the second visual indicator in real-time as the ultrasound probe is maneuvered from the current position into the first position.

35. The method of claim 20, further comprising:
in the second mode:
determining that the current position of the ultrasound probe has reached alignment with the first position of the ultrasound probe in accordance with predetermined alignment criteria; and
acquiring second ultrasound image data from the ultrasound probe, while the ultrasound probe is in alignment with the first position of the ultrasound probe.

36. The method of claim 35, further comprising:
in accordance with a determination that the current position of the ultrasound probe is in alignment with the first position of the ultrasound probe, associating the second ultrasound image data with the first ultrasound image data as image data taken using the same probe position.

37. The method of claim 36, further comprising:
recording probe alignment information associated with acquisition of the second ultrasound image data; and
utilizing the probe alignment information in image registration between the first ultrasound image and the second ultrasound image data.
